(19) United States
(12) Patent Application Publication
(10) Pub. No.: US 2023/0078524 A1
(43) Pub. Date: Mar. 16, 2023

(54) RAPID GENETIC SCREENING METHOD AND DEVICE

(71) Applicant: Changzhou Trendi Medical Technology Co., Ltd, Changzhou City (CN)

(72) Inventors: Kwok Leung Patrick Kwan, Changzhou City (CN); Qiaoling Chen, Changzhou City (CN)

(21) Appl. No.: 17/739,072

(22) Filed: May 6, 2022

(30) Foreign Application Priority Data
Sep. 10, 2021 (CN) 202111060683.6

(51) Int. Cl. C12Q 1/6825; G01N 27/327; B01L 3/00

(52) U.S. Cl. CPC C12Q 1/6825; G01N 27/3276; B01L 3/502738; B01L 2400/06; B01L 2200/10; B01L 2300/0645; B01L 2300/0816; B01L 2300/18

(57) ABSTRACT

The present disclosure relates to a rapid genetic screening method and device. The method includes: collecting a sample to be tested of a patient through a micro-fluidic chip, where the sample to be tested includes a whole blood or saliva or nasopharyngeal swab or wound swab sample of a patient; lysing and amplifying the sample to be tested in the micro-fluidic chip to obtain an amplified nucleic acid segment; fusing a biosensor with amplification liquid, where the biosensor is provided with a DNA probe which can only be bounded to a specific nucleic acid segment and in which an impedance may dramatically change before and after the bounding; and inputting an electrical signal to the biosensor, testing a signal of an output end, and determining whether a nucleic acid segment matched with the DNA probe exists in the sample to be tested of the patient. The DNA probe can be replaced to test whether different nucleic acid segments exist. A person only need to collect the sample to be tested of the patient, select a probe, and configure simple parameters, so that the operations are simple, without performing nucleic acid extraction and purification on the sample to be tested, and the testing efficiency is greatly improved.

### TECHNICAL FIELD

The present disclosure relates to the technical field of precision medicine, in particular to a rapid genetic screening method and device.

### BACKGROUND ART

Precision medicine is an emerging method of disease prevention and treatment that takes personal genes, environment, and difference in living habits into account. It is a new-type medical concept and medical mode developed on the basis of individualized medicine with the rapid advancement of a genomic test technology and cross application of biological information and big data science. Its essence is to analyze, identify, verify, and apply biomarkers for large sample populations and specific disease types through genome, proteome and other omics technologies and cutting-edge medical technologies, so as to accurately find the cause of a disease and a target of treatment, and different states and processes of a disease are accurately classified, thus ultimately achieving the purpose of personalized and precise treatment for diseases and specific patients, and improving the benefits of disease diagnosis and prevention. Precision medicine includes two aspects: diagnosis and treatment. “Precision” is the core, and gene test is the foundation. The causes of the same type of diseases may be different. For example, the cause of lung cancer may be the mutation of one of the factors such as EGFR, K-RAS, ROSIGF, and C-MET. Lung cancers caused by different causes require drugs in different quality and quantities. AKT/PI3K inhibitors are selected for the lung cancer caused by K-RAS mutation, and TKIs+ chemotherapy is better for the lung cancer caused by EGFR mutation. Gene test is the basis for accurate discovery and diagnosis of causes. In a true precision medicine market, clinicians need to be the main body for applying gene test to the clinic. Precisely because of the vigorous development of precision therapy, the current clinical demand for Point-of-care Testing (POCT) of genes has greatly increased.

In the prior art, traditional gene testing generally uses a fluorescent PCR (Polymerase Chain Reaction) technology, which need to be performed in several steps and difficult to be fully automatic, causing the process complex and professionalized, because the demands of nucleic acid extraction and purification. The preparation of PCR fluorescence quantification requires professional skills and people, makes it only can be carried out in a laboratory and difficult to be used for clinical testing and on-site testing. Meanwhile, the detection period of this technical method is long. Because professionals and professional institutions are required for testing, most of the tests can only be carried out in qualified laboratories and institutions. Samples need to be collected from hospitals and then sent to a professional testing institution. After a batch of samples is collected, batch PCR amplification is then performed; and testing results are sent to the hospitals. The entire process from sample collection to obtaining of a testing result report takes days or even a week. This is the standard method used in clinical diagnosis. Since performing the test takes long time, Point of Care Testing (POCT) cannot be realized. The patient cannot get the results at the time when they visit the doctor, and needs to make a second doctor appointment a few days later to get the result, which increases the workload of a doctor, and the patient's consultation cycle and expenses. Furthermore, for some emergency treatments that require immediate testing results, the treatment for the disease will also be delayed.

### SUMMARY

The present disclosure provides a rapid genetic screening method and device, so as to solve the problems of long testing period, which increases of testing costs of a doctor and a patient, and a delay in treatment due to the fact that the genetic testing process in the prior art is complex and requires a professional institution and professionals.

The above objective of the present disclosure is achieved through the following technical solutions:

In a first aspect, an embodiment of the present disclosure provides a rapid genetic screening method, including:

receiving a sample to be tested, and lysing the sample to be tested at a high temperature to obtain cell lysate including a nucleic acid, so the sample to be tested could be collected as a whole blood or saliva or nasopharyngeal swab or wound swab sample of a patient;

performing isothermal amplification on the cell lysate by means of a LAMP principle to obtain amplified nucleic acid liquid product;

fusing the nucleic acid liquid product with a preset biosensor, where the biosensor is pre-immobilized with a DNA probe which is only bounded to the specific nucleic acid segment, and after the DNA probe is bounded to the specific nucleic acid segment, the biosensor changes a self impedance value;

inputting an electrical signal to an input end of the biosensor, and collecting signal data of an output end of the biosensor; and

determining, on the basis of the signal data, whether the sample to be tested contains a target nucleic acid segment matched with the DNA probe.

Further, the determining, on the basis of the signal data, whether the sample to be tested contains a target nucleic acid segment matched with the DNA probe includes:

constructing a machine-learning algorithm model;

extracting a characteristic value of the signal data of the output end of the biosensor by means of a characteristic extraction algorithm combined with preset principal component analysis and a support vector machine; and

determining, by means of a preset machine-learning algorithm model and the characteristic value, whether the sample to be tested contains a target nucleic acid segment matched with the DNA probe.

Further, the method further includes: judging, by means of a preset hyperplane dichotomy algorithm, a determination result of whether the sample to be tested contains a target nucleic acid segment matched with the DNA probe.

Further, the method further includes: eliminating, on the basis of a preset algorithm model, signal noise generated by impurities in the nucleic acid liquid product.

In a second aspect, an embodiment of the present disclosure further provides a rapid genetic screening device, including: a micro-fluidic chip, a biosensor, and a portable testing main body, wherein the micro-fluidic chip is detachably inserted on the portable testing main body; and the biosensor is arranged on the micro-fluidic chip.

The micro-fluidic chip includes a first chamber, a second chamber, and a third chamber; the first chamber is used for loading a sample to be tested; the second chamber is used for performing loop-mediated isothermal amplification (LAMP) reaction; the third chamber is provided with the biosensor and is used for fusion of nucleic acid liquid product with the biosensor; a first micro valve is arranged between the first chamber and the second chamber; a second micro valve is arranged between the second chamber and the third chamber.

The portable testing main body includes a main control module, and an interface module, a temperature control module, a power control module, a frequency generation module, a data storage module, and a signal processing/AI algorithm module which are all connected to the main control module.

The main control module is connected to the micro-fluidic chip through the interface module, and controls, by means of the temperature control module, a temperature of each chamber of the micro-fluidic chip; flowing of liquid inside the micro-fluidic chip and opening and closing of the micro valves are controlled by means of the power control module; an electrical signal is generated by the frequency generation module and is input into the biosensor; signal data of an output end of the biosensor is stored and recorded by means of the data storage module; and the signal data of the output end of the biosensor is analyzed by means of the signal processing/AI algorithm module.

Further, the power control module includes a motor driving sub-module, a micro pump sub-module, and a flow control sub-module.

The motor driving sub-module, the micro pump sub-module, and the flow control sub-module are all connected to the main control module.

Further, the interface module includes a plurality of USB interfaces.

The plurality of USB interfaces is used for being simultaneously connected to a plurality of micro-fluidic chips.

Further, the device further includes a testing chip gating/control module.

The main control chip selects and controls, through the testing chip gating/control module, the micro-fluidic chip connected to the portable testing main body.

Further, the device further includes a communication module.

The communication module is connected to the main control module and is used for transmitting data to external equipment.

Further, the device further includes a human-computer interaction module.

The technical solutions provided by the embodiments of the present disclosure can include the following beneficial effects.

In the technical solution provided by the embodiments of the present disclosure, the sample to be tested of the patient is collected by means of the preset micro-fluidic chip; heating, lysing and isothermal amplification are performed on the sample to be tested in the micro-fluidic chip to obtain an amplified nucleic acid segment; the biosensor is fused with the amplified nucleic acid segment, where the biosensor is pre-immobilized with the DNA probe which can only be bounded to a specific nucleic acid segment and in which an impedance value may dramatically change before and after the bounding to the specific nucleic acid segment; the electrical signal is input to an input end of the biosensor; whether the nucleic acid segment that matched with the DNA probe exists in the sample to be tested of the patient can be determined by testing the signal data of the output end of the biosensor; and whether different nucleic acid segments exist in the blood sample of the patient can be tested by changing the complementary DNA probe. In this process, a person only needs to collect the sample to be tested, select the DNA probe, and correspondingly configure simple parameters, so that the operations are simple, and the requirement for professional skills is low; at the same time, it is not necessary to transfer the sample to a laboratory or a test institution, so that the testing efficiency is improved, and POCT is achieved; and the testing costs of a doctor and a patient are reduced.

It should be understood that the above general description and the following detailed description are exemplary and explanatory only, and are not intended to limit the present disclosure.

### BRIEF DESCRIPTION OF THE DRAWINGS

The accompanying drawings, which are incorporated in and constitute a part of this specification, illustrate examples consistent with the present disclosure and, together with the specification, serve to explain the principles of the present disclosure.

FIG. 1 is a flow chart of genetic testing in the prior art;

FIG. 2 is a flow chart of a rapid genetic screening method provided by an embodiment of the present disclosure;

FIG. 3 is a schematic structural diagram of a rapid genetic screening device provided by an embodiment of the present disclosure;

FIG. 4 is a schematic diagram of an appearance of a rapid genetic screening device provided by an embodiment of the present disclosure;

FIG. 5 is a schematic structural diagram of a rapid genetic screening device provided by another embodiment of the present disclosure;

FIG. 6 is a flow chart of use of a rapid genetic screening device provided by an embodiment of the present disclosure.

1: portable testing main body; 2: micro-fluidic chip; 3: biosensor; 4: main control module; 5: interface module; 6: temperature control module; 7: power control module; 8: frequency generation module; 9: data storage module; 10: signal processing/AI algorithm module; 11: testing chip gating/control module; 12: human-computer interaction module; 13: power management module; and 14: communication module.

### DETAILED DESCRIPTION OF THE EMBODIMENTS

Exemplary examples will be described in detail herein, examples of which are illustrated in the accompanying drawings. When the following description refers to the accompanying drawings, the same numbers in different drawings represent the same or similar elements unless otherwise indicated. The implementation modes described in the following exemplary embodiments do not represent all embodiments consistent with the present disclosure. Rather, they are merely examples of devices and methods consistent with some aspects of the present disclosure as detailed in the appended claims.

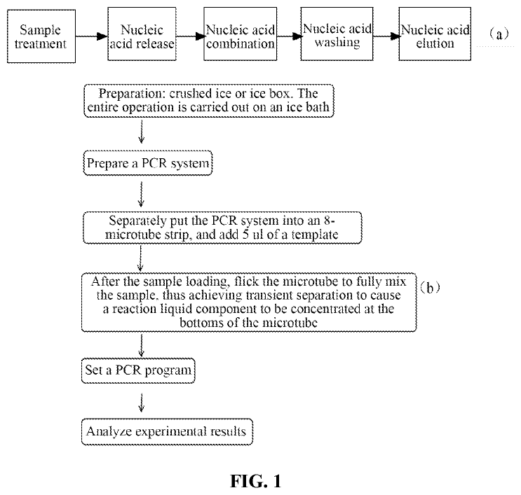

FIG. 1 is a flow chart of genetic testing in the prior art. As shown in FIG. 1, a fluorescent PCR technology is used for the current traditional genetic testing. In the technology, a testing result is extracted from a sample. First of all, steps in FIG. 1(a) need to be carried out: general sample treatment, nucleic acid release, nucleic acid binding, nucleic acid washing, and nucleic acid elution. For example, extracting nucleic acid by a centrifugal column method includes: sample collection, lysing, washing, elution, and obtaining of purified nucleic acid for later use. The flow in FIG. 1(b) then needs to be carried out, including: 1. Crushed ice or an ice box is prepared. The entire process is carried out in an ice bath. 2. A PCR system is prepared. A 50 ul system is taken for example here, including: 38 ul of a qPCR buffer, 0.4 ul of an upstream primer, 0.4 ul of a downstream primer, 0.2 ul of a probe, 2 ul of an enzyme mixed solution, and ddH2O supplemented to 45 ul. 3. 5 ul of a template is added into a separated 8-microtube strip. 4. After the sample loading, the microtube is flicked to fully mix the sample, thus achieving transient separation to cause a reaction liquid component to be centralization at the bottoms of the microtube. 5. A PCR program is set. An RNA template is taken for example, including 10 min at 50° C., pre degeneration of cDNA for 3 min at 95°, degeneration for 15 s at 95°, annealing at 60° C., extending and fluorescence collection for 30 s, and degeneration, annealing and the like for 40 cycles. 6. Experimental results are analyzed, including: 1, the abscissa of an amplification curve represents the number of amplification cycles, and the ordinate represents the fluorescence intensity; a fluorescence signal is collected once in each cycle. 2. A Ct value is obtained according to the number of cycles when the fluorescence signal of an amplification product reaches a preset threshold during the PCR amplification. The Ct value is highly reproducible.

A complex reagent preparation operation and a process operation are included. The above traditional fluorescent PCR technology has the following disadvantages:

1. The operation is step-by-step, non-automatic, and requires professionals, such as extraction and purification of nucleic acid, and preparation for PCR fluorescence quantification. Therefore, the operation can only be performed in a laboratory and cannot be used for clinical testing and on-site testing.

2. The detection period is long. Because professionals and professional institutions are required for testing, most of the tests can only be carried out in qualified laboratories and testing institutions. Samples need to be collected from hospitals and then sent to a professional testing institution. After a batch of samples is collected, batch PCR amplification is then performed; and testing results are sent to the hospitals. The entire process from sample collection to obtaining of a testing result report takes days or even a week. In clinical diagnosis, since POCT cannot be realized, the patient cannot get the results at the time when they visit the doctor, and needs to make a second doctor appointment a few days later to get the result, which increases the workload of a doctor, and the patient's consultation cycle and expenses. For some emergency treatments that require immediate testing results, great impact will be caused.

3. The equipment has a large volume and is expensive, so that it is generally only equipped in top three hospitals and professional testing institutions. It can be seen from the genetic testing technical solution of fluorescent PCR that several different instruments are required to complete extraction and purification of sample nucleic acid to PCR amplification and testing, such as a nucleic acid extractor, a PCR amplification instrument, and fluorescence testing equipment. The steps, the principle, and the design are complicated, so each kind of equipment is huge and expensive. For example, the price of an automatic nucleic acid extractor ranges from thousands RMB to tens of thousands RMB; the price of the per amplification instrument ranges from tens of thousands RMB to hundreds of thousands RMB; and the price of the fluorescence tester is tens of thousands RMB.

With the rapid development of precision medicine, POCT has become a trend, and the miniaturization and microminiaturization of genetic testing equipment also become a trend. Although the industry is also committed to a miniaturization design of this traditional fluorescent PCR testing technology, because of the principle of the PCR technology, multiple low and high-temperature cycles are required, and repeated heating and cooling need to be performed on a tiny chip, so that there are many bottlenecks that need to be broken through for engineering implementation, and a lot of experiments are required. Second, a fluorescence-based identification solution requires the support of a laser source and a complex optical system, and is not suitable for miniaturized and low-cost clinical applications. In addition, complex nucleic acid extraction and other steps are also required, so it has not been possible to achieve miniaturization based on the fluorescent PCR technology so far. Fast and fully automatic testing equipment can be used in clinical applications to achieve rapid on-site genetic screening, and realize seamless testing at any time anywhere for genetic screening.

At present, there is a large flow of patients in Chinese hospitals, and clinicians need to quickly obtain testing results and provide treatment plans for patients, so as to reduce the difficulty of seeking medical advices for the patients. Moreover, for some emergent diseases that threaten the life safety of patients, accurate treatment needs to be immediately and effectively provided for patients according to rapid screening results, and before some emergency surgical operations, testing results of certain diseases of the patients also need to be obtained in time. The traditional genetic testing methods mentioned above cannot meet the requirements.

In order to solve the above problems, the present disclosure provides a rapid genetic screening method and device, so as to solve the problems of long testing period, which increases of testing costs of a doctor and a patient, and a delay in treatment due to the fact that the genetic testing process in the prior art is complex and requires a professional institution and professionals, and improve the clinical genetic testing efficiency. Specific implementation solutions are described in detail through the following embodiments.

### Embodiments

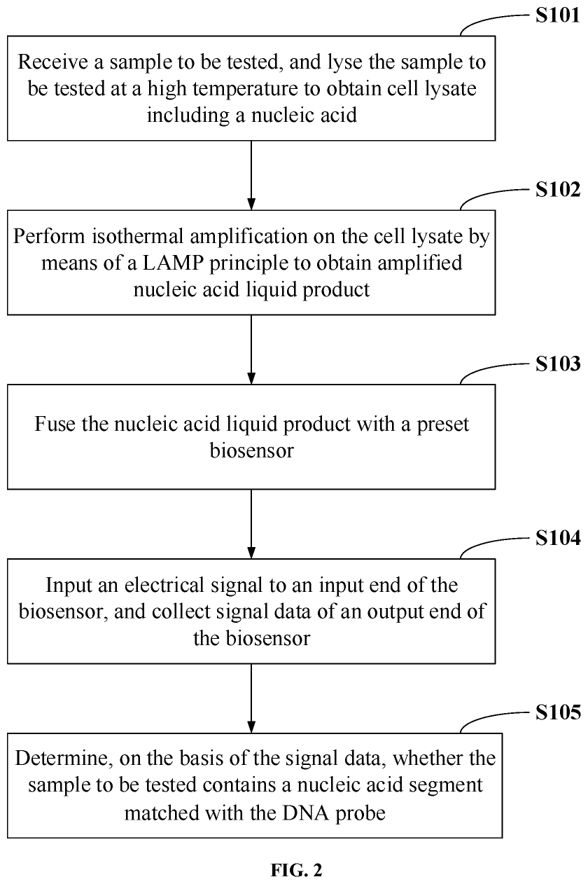

FIG. 2 is a flow chart of a rapid genetic screening method provided by an embodiment of the present disclosure. As shown in FIG. 2, the method at least includes the following steps:

S101, a sample to be tested is received, and is lysed at a high temperature to obtain cell lysate containing a nucleic acid.

The sample to be tested includes a whole blood sample or a saliva sample or a nasopharyngeal swab sample or a wound swab sample of a patient. The whole blood sample is described below. However, it can be understood that this is only for full description of its principle, and is not intended to limit the specific protection scope. In actual applications, in the rapid genetic screening method provided by the present disclosure, the above sample can also be sampled to complete the genetic testing.

Specifically, the blood of the patient and the above sample can be tested through a micro-fluidic chip. The micro-fluidic chip includes a plurality of chambers. The sample to be tested of the patient is loaded in the first chamber. After completing the sample loading, the medical staff plugs the micro-fluidic chip with the sample to be tested into a preset portable testing main body. The preset portable testing main body controls the temperature of the micro-fluidic chip, and heats the micro-fluidic chip. When the micro-fluidic chip is heated to a preset temperature such as 95° C., the sample to be tested is lysed at the high temperature. After preset time, such as 5 min, heating is stopped, thus obtaining cell lysate with nucleic acid template.

In actual applications, the micro-fluidic chip can also be directly fixed on the preset portable testing main body. When the medical staff collect blood, liquid to be tested collected from the patient can be placed on a sample collection port of testing chip, i.e., the micro-fluidic chip, thus completing sample collection.

S102, isothermal amplification is performed on the cell lysate by means of a LAMP principle to obtain amplified nucleic acid liquid product.

Specifically, after the above cell lysate is obtained, the cell lysate will be transferred into the second chamber automatically controlled by the portable testing main body, for example, opening a micro valve between the first chamber and the second chamber through in the microfluidic chip to enable the cell lysate move into the second chamber, and then controls automatically, through the portable testing main body, the temperature of the second chamber to provide a reaction environment for LAMP. For example, the second chamber is heated through the portable testing main body to 65° C., and the temperature is maintained for 30 min. A LAMP reaction mixture is pre-loaded in the second chamber or stored in another independent chamber connected to the second chamber, such as a primer and LAMP reaction enzymes. After the above cell lysate moves into the second chamber, and the second chamber reaches a reaction temperature, the primer and the various enzymes react with the cell lysate in the second chamber to obtain amplified nucleic acid liquid product after LAMP.

It should be noted that in the above-mentioned LAMP reactant, some primers and LAMP reaction enzymes cannot be mixed. In the rapid genetic screening method provided by the present disclosure, reactants that cannot be mixed, such as the primer or the enzyme, can be disposed in respective sub-chambers in the second chamber, or in other independent chambers connected to the second chamber. When it is necessary to perform the LAMP reaction, a plurality of micro valves, such as a primer micro valve, an enzyme, and other chemical mixture micro valves, are controlled through the portable testing main body to enable the reactants to flow into the second chamber and mix with the cell lysate for LAMP reaction, thus ensuring normal reaction.

S103, the nucleic acid liquid product is fused on the preset biosensor.

The biosensor is pre-provided with a DNA probe which is only bounded to a specific target nucleic acid segment, and after the DNA probe is bounded to the specific nucleic acid segment, the biosensor changes a self impedance value.

Specifically, after the nucleic acid liquid product of isothermal amplification is obtained, a preset micro valve is controlled through the portable testing main body to enable the nucleic acid liquid product flow into the third chamber. In the third chamber, the nucleic acid liquid product is fully fused with the biosensor. It should be noted that the biosensor can adopt an interdigital electrode (IDE) biosensor. The IDE biosensor is pre immobilized with a DNA probe. The DNA probe can be only bounded to the specific nucleic acid segment, that is, the probe can be only bounded to a nucleic acid segment matched with the probe. After the probe is bounded to the target nucleic acid segment, the impedance characteristic of the IDE biosensor will be affected, and the impedance of the IDE biosensor will dramatically change. After the above nucleic acid liquid product flows into the third chamber, the IDE biosensor in the third chamber is automatically fused with the nucleic acid liquid product. After the fusion, if the nucleic acid liquid product contains the nucleic acid segment matched with the probe on the chip, the probe will be automatically bounded to the nucleic acid segment. At this time, the impedance of the IDE biosensor dramatically changes. If the nucleic acid liquid product does not contain the nucleic acid segment matched with the probe on the chip, the probe will not be bounded to a nucleic acid segment. At this time, the impedance of the IDE biosensor does not dramatically change.

That is, if the sample to be tested of the patient is positive, and contains a nucleic acid segment to be tested, the nucleic acid segment in the amplified nucleic acid liquid product will be fully bounded to the probe on the IDE biosensor, and the impedance characteristic of the IDE biosensor will dramatically change. After an electrical signal is input to an input end of the IDE biosensor, an amplitude and phase of a signal of an output end of the IDE biosensor will both dramatically change. If the sample to be tested of the patient is negative, after the amplified nucleic acid liquid product reacts with it, the obtained nucleic acid liquid product does not contain the nucleic acid segment to be tested, and the impedance characteristic of the IDE biosensor does not change too much. The amplitude and phase of the signal of the output end of the IDE biosensor does not dramatically change.

S104, an electrical signal is input to the input end of the biosensor, and signal data of the output end of the biosensor is collected.

Specifically, in the fusion process of the nucleic acid liquid product and the IDE biosensor or before and after the fusion, the electrical signal is input to the input end of the IDE biosensor. For example, a series of analog voltage signals with the amplitude of 20 my are input to the input end of the IDE biosensor through the portable testing main body in a sweep mode. Start and stop frequency points of sweep are configured by system software parameters according to an actual debugging condition, within a maximum range from 100 HZ to 100 KHZ. A step length is configured according to an actual product and experimental requirement software. A minimum step length is 1 HZ, and a maximum step length is 1 KHZ. The signal data of the output end of the IDE biosensor is obtained by collection.

In actual applications, the electrical signal can be input to the input end of the IDE biosensor before the nucleic acid liquid product is fused on the IDE biosensor. After preset time of fusion of the nucleic acid liquid product and the IDE biosensor, whether the nucleic acid liquid product contains the nucleic acid segment matched with the probe is determined by collecting the output signal data of the IDE biosensor before and after the fusion.

S105, whether the sample to be tested contains the nucleic acid segment matched with the DNA probe is determined on the basis of the signal data.

Specifically, since the impedance of the IDE biosensor may dramatically change after the probe is bounded to the specific nucleic acid segment, whether the nucleic acid liquid product contains the nucleic acid segment matched with the probe on the IDE biosensor can be determined by collecting the signal data of the output end of the IDE biosensor and observing whether the impedance value of the IDE biosensor changes, thus realizing collection of a sample of a patient and determining whether the patient has a target nucleic acid segment of a certain disease, which realizes genetic testing and screening.

In actual applications, after the above reaction, whether the sample to be tested contains the nucleic acid segment matched with the probe can be determined more accurately through a machine-learning algorithm model, a characteristic extraction algorithm, and the like.

Specifically, it may be found through an impedance characteristic curve graph formed by the series of sweep points that the characteristic values of the impedance characteristic curve graphs of positive patients are similar and centralization, while the characteristic values of the impedance characteristic curve graphs of negative patients are similar and centralization. The machine-learning algorithm model is used to process data. The characteristic extraction algorithm combined with principal component analysis and a support vector machine is used to comprehensively process results of four quadrants (the real part, the virtual part, the amplitude, and the phase), so as to determine a classification result. Furthermore, a judgment can also be made through a hyperplane dichotomy algorithm, so as to improve the sensitivity and the specificity.

According to the rapid genetic screening method provided by the embodiment of the present disclosure, by means of the LAMP technology, in combination with the micro-fluidic chip technology, the IDE biosensor device modified by the DNA probe is used to convert a chemical signal of a nucleic acid amplification result into an electrical signal characterization. The measured electrical signal is processed and artificially and intelligently analyzed through the handheld portable testing main body, so that accurate genetic testing is achieved.

Further, the rapid genetic screening method further includes: signal noise generated by impurities in the nucleic acid liquid product is eliminated on the basis of a preset algorithm model.

Specifically, in this method, extraction and purification of nucleic acid are not required. In the above process, after cell lysate is mixed with the primers and other reaction reagents for amplification, some chemical impurities will be generated. These chemical impurities have no coupling characteristic with the IDE biosensor, so that electrical signal characteristics shown by the chemical impurities are some signal noises. These signal noises are processed by the algorithm model in the prior art, so that the influence of the chemical impurities on useful signals can be reduced, and the judgment accuracy is further improved, thus improving the sensitivity and specificity.

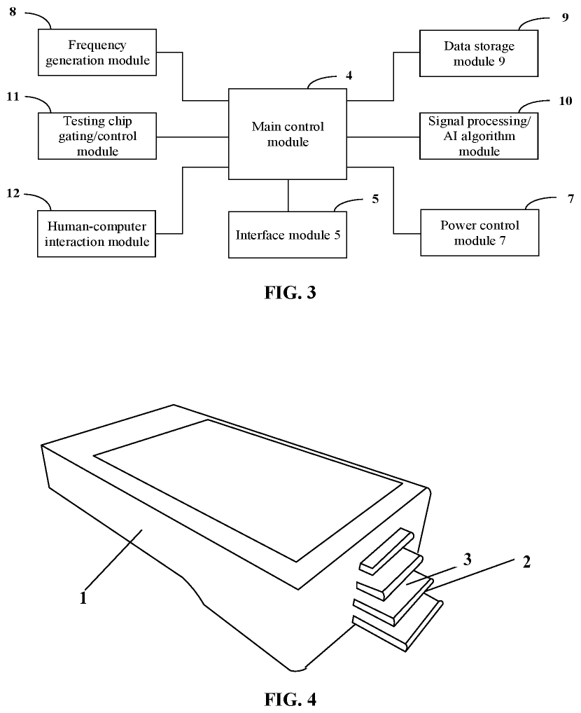

Based on the same inventive concept, the present disclosure further provides a genetic screening device. FIG. 3 is a schematic structural diagram of a rapid genetic screening device provided by an embodiment of the present disclosure, and FIG. 4 is a schematic diagram of an appearance of a rapid genetic screening device provided by an embodiment of the present disclosure. As shown in FIG. 3 and FIG. 4, the rapid genetic screening device provided by the embodiment of the present disclosure includes a portable testing main body 1, a micro-fluidic chip 2, and a biosensor 3. The micro-fluidic chip 2 is detachably plugged on the portable testing main body 1, and the biosensor 3 is arranged on the micro-fluidic chip 2.

The micro-fluidic chip 1 includes a first chamber, a second chamber, and a third chamber; the first chamber is used for loading a sample to be tested; the second chamber is used for performing LAMP; the third chamber is used for storing the biosensor 2; a micro valve is arranged between the first chamber and the second chamber; and a micro valve is arranged between the second chamber and the third chamber. In addition, a third micro valve and a fourth micro valve are also arranged between a chamber used for storing a LAMP reactant alone and the second chamber.

The portable testing main body 1 includes a main control module 4, and an interface module 5, a temperature control module 6, a power control module 7, a frequency generation module 8, a data storage module 9, and a signal processing/AI algorithm module 10 which are all connected to the main control module.

The main control module 4 is connected to the micro-fluidic chip 2 through the interface module 5, and controls, by means of the temperature control module 6, a temperature of each chamber of the micro-fluidic chip 2; flowing of liquid inside the micro-fluidic chip 2 is controlled by means of the power control module 7; an electrical signal is generated by the frequency generation module 8, such as an AD5933 circuit module, and is input into the biosensor 3; signal data of an output end of the biosensor 3 is stored and recorded by means of the data storage module 9; and the signal data of the output end of the biosensor 3 is analyzed by means of the signal processing/AI algorithm module 10.

Further, the power control module 7 includes a motor driving sub-module, a micro pump sub-module, and a flow control sub-module; and the motor driving sub-module, the micro pump sub-module, and the flow control sub-module are all connected to the main control module 4. Under the control of the main control module 4, the working state of the micro-fluidic chip 2 is controlled, such as controlling flow-in of primers and enzymes therein, and opening and closing of the micro valves.

In some specific implementation processes, the rapid genetic screening device provided by the present disclosure further includes a testing chip gating/control module 11 and a human-computer interaction module 12 which are connected to the main control module 4. Furthermore, the interface module 5 includes a plurality of USB interfaces used for being respectively connected to a plurality of micro-fluidic chips, and a communication interface used for being connected to external equipment, such as an external mobile phone and a computer. The states of the portable testing main body 3 and the plurality of micro-fluidic chips are controlled through the testing chip gating/control module 11, so that the rapid genetic screening device becomes a multi-channel signal testing instrument which can simultaneously test various pieces of testing chip, i.e., the plurality of micro-fluidic chips. The testing chip may be a testing chip for testing the same gene from different patient samples, or may be a testing chip for testing different genes from the same patient sample or different patient samples. Medical staff can configure, in real time by means of the human-computer interaction module 12, different channels to test various types of genes, configure parameters, and read data. The human-computer interaction module 12 can include an interaction display interface, such as a touch screen, which can display a testing progress in real time and acquire testing results, thus realizing multi-channel multi-site genetic testing and improving the testing and screening efficiency.

In addition, a plurality of chambers can also be provided in the micro-fluidic chip 2, and reagents that cannot be mixed in the reaction process can be stored in different chambers, and an independent waste liquid collection chamber and the like are provided to ensure accurate and efficient internal reactions.

In actual applications, the biosensor can adopt an IDE biosensor. The IDE biosensor modified by a DNA probe can be integrated in the independent chamber in the micro-fluidic chip, so that the device is more stable and reliable. During testing and screening of different genes, the IDE biosensor is replaced.

It should be noted that in the rapid genetic screening device provided by the present disclosure, the chambers in the micro-fluidic chip need to be heated. A specific heating manner may be as follows: A heating device and a temperature measurement device may be integrated in the micro-fluidic chip 2, and cooperate with the temperature control module 6 and the main control module 4 in the portable testing main body 1 to control the temperature of each chamber. The heating device and the temperature measurement device can also be integrated at specific positions on the portable testing main body 1. After the micro-fluidic chip 2 is plugged to the portable testing main body 1, under the control of the main control module 4 and the temperature control module 6, each chamber in the micro-fluidic chip 2 is heated.

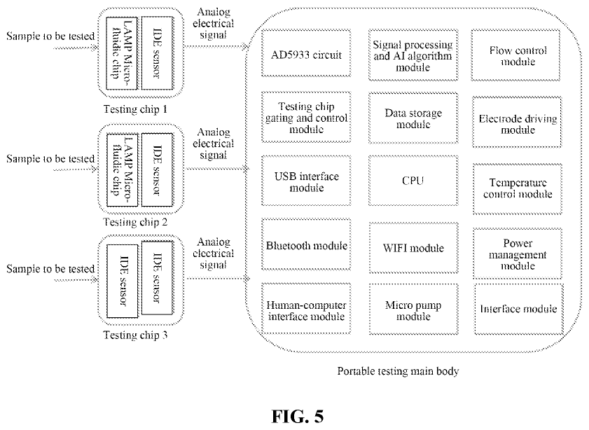

FIG. 5 is a schematic structural diagram of a rapid genetic screening device provided by another embodiment of the present disclosure. As shown in FIG. 5, in the rapid genetic screening device provided by the embodiment of the present disclosure, the main control module 1 may be achieved by a central processing unit (CPU). In addition to the circuit structure mentioned in the above device embodiment, a power management module 13 connected to the CPU and used for managing power supply of the device, and a communication module 14 used for communicating with and transmitting data to the external equipment and including a WIFI sub-module, a Bluetooth sub-module, and the like are further included.

The rapid genetic screening device provided by the present disclosure is a system-level technology. By means of the joint cooperation of the reagents, the testing chip, i.e., the micro-fluidic chip, the testing instrument, i.e., the portable testing main body, and application software arranged inside the portable testing main body and on other external equipment, a rapid genetic screening function is completed. For different genetic testing, the micro-fluidic chip and the portable screening device are both reusables, as long as reagents and the DNA probe on the biosensor are disposable, and in the screening process, targeted parameters are configured for the device.

After a testing chip and a DNA probe are selected, and relevant parameters are configured, the device automatically realizes flow control, temperature control, automatic data collection, and automatic micro valve control, without operations from professionals. In the testing process, a whole blood sample or other samples to be tested are used. Extraction and purification of nucleic acid are not required, so that the operation is simple, and targeted testing is achieved. It only takes half an hour to one hour from sample loading to obtaining the results, so that the testing efficiency is greatly improved. Furthermore, due to the integrated design of the micro-fluidic chip and the IDE biosensor, the device is minimized. By the multi-channel design, on-site multi-site genetic testing outside a laboratory can be carried out.

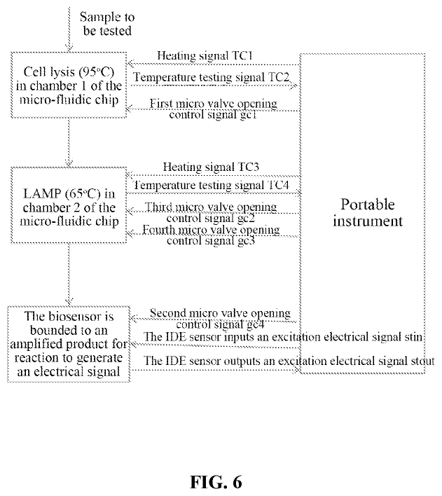

In order to describe the rapid genetic testing and screening device provided by the embodiment of the present disclosure more clearly, a specific use process of the device is schematically illustrated. FIG. 6 is a flow chart of use of a rapid genetic screening device provided by an embodiment of the present disclosure. As shown in FIG. 6, medical staff loads a sample collected from a patient at a sample collection port of micro-fluidic testing chip, and the rapid genetic screening device can start to perform genetic screening automatically, without any manually control of the medical staff during the process.

TC1 is a heating signal transmitted by an instrument to chamber 1 of the micro-fluidic chip, that is, the first chamber; TC2 is a temperature testing signal transmitted by the micro-fluidic chip to chamber 1 of the instrument; TC3 is a heating signal transmitted by the instrument to chamber 3 of the micro-fluidic chip, that is, the third chamber; TC4 is a temperature testing signal transmitted by the micro-fluidic chip to chamber 2, that is the second chamber; gc1 is a one-way valve control signal from the first chamber of the micro-fluidic chip to the second chamber; gc2 is a one-way valve control signal from a primer storage area of the micro-fluidic chip to the second chamber; gc3 is a one-way valve control signal from a liquid area of the micro-fluidic chip for storing enzymes and other nucleic acid liquid products to the second chamber; Gc4 is a one-way valve control from the first chamber of the micro-fluidic chip to the biosensor; stin is an input excitation electrical signal from the instrument to the IDE sensor by the instrument; and stout is an electrical signal output by the IDE sensor to the instrument.

The sample to be tested is dripped or added. A blood sample is taken as an example. After the blood is dripped, the testing instrument is turned on, that is, the portable testing main body starts to work. The instrument first sends the heating signal TC1 to the first chamber of the micro-fluidic chip for heating. The first chamber contains the loaded blood sample and a diluent, and cell lysis is carried out at a temperature of 95° C. to release nucleic acids. The temperature testing signal TC2 of the first chamber is sent to the portable testing main body in real time. Whether the temperature has risen to 95° C. is determined according to a relation table for the corresponding TC2 signal and temperature inside the instrument. If there is a large difference, the step length of the heating signal TC1 is increased to indicate fast heating. If the temperate is close to 95° C., the heating step length is reduced for fine adjustment. Through the cooperation of the two signals TC1 and TC2, the temperature of the first chamber is maintained at about 95° C. for 5 min, and a temperature error is controlled to be −1° C. to 1° C., so that the cells are fully lysed to release the nucleic acid required for subsequent isothermal amplification. After cell lysis is completed in the first chamber of the micro-fluidic chip (which is determined on the basis of maintaining 95° C. for 5 min), the testing main body sends an open signal gcl of the first micro valve to enable the cell lysate to enter, according to a required flow, the second chamber, that is, chamber 2, of the micro-fluidic chip for LAMP; the instrument sends signals to open the third micro valve and the fourth micro valve in sequence, so that the primers and other chemical substances required for amplification stored in a micro-fluidic storage area accurately enter the second chamber according to required flows; next, the instrument sends the heating signal TC3 to the second chamber of the micro-fluidic chip for heating; at the same time, a temperature control circuit of the second chamber feeds back a temperature testing signal TC4 to the testing main body; by cooperation of the two signals cooperate, after the temperature of the second chamber reaches 65° C., the temperature is maintained for about 30 min; a temperature error range is −1° C. to 1° C.; full LAMP reaction is performed, so that the required nucleic acid segment is fully amplified. After 30 min (this value can be changed according to an actual experiment after parameters are reconfigured to a register by software) of amplification, the testing main body initiates a signal to open the second micro valve to allow the fully amplified nucleic acid liquid product to flow to the biosensor, and the nucleic acid liquid product and the biosensor are fully combined. After the reaction, by the above device and the method mentioned in the method embodiment, the signal data of the output end of the IDE biosensor is automatically collected, and data analysis is automatically performed on the basis of the signal data to obtain testing and screening results.

The rapid genetic screening device provided in the embodiment of the present disclosure has the following advantages: The nucleic acid extraction, amplification, and testing of the sample are fully automatically and integrally completed, without the need for professionals; it is fast, and can be completed in 30-60 min from sample loading to displaying of testing results; it has a wide range of applications and can be used both inside and outside the laboratory for on-site testing; the equipment is small in size and cheap, which is beneficial for clinical applications; and in addition to the top three hospitals, it is suitable for temporary testing sites, temporary hospitals, grass-roots hospitals, community hospitals, physical examination and health monitoring institutions, etc.

It can be understood that the same or similar parts in the foregoing embodiments may be referred to each other, and the contents not described in detail in some embodiments may refer to the same or similar contents in other embodiments.

It should be noted that in the description of the present disclosure, the terms “first”, “second”, etc. are only used for descriptive purposes, and cannot be understood as indicating or implying relative importance. In addition, in the description of the present disclosure, unless otherwise specified, the meaning of “plurality” means at least two.

Any process or method description in the flowchart or described in other ways herein can be understood as a module, segment or part of a code that includes one or more executable instructions for implementing specific logical functions or steps of the process. The scope of the preferred embodiments of the present disclosure includes additional implementations, which may not be in the order shown or discussed, including performing functions in a substantially simultaneous manner or in the reverse order according to the functions involved. This should be understood by those skilled in the art to which the embodiments of the present disclosure belong.

It should be understood that each part of the present disclosure can be implemented by hardware, software, firmware or a combination thereof. In the above implementation modes, multiple steps or methods can be implemented by software or firmware stored in a memory and executed by a suitable instruction execution system. For example, if it is implemented by hardware, as in another implementation, it can be implemented by any one or a combination of the following technologies known in the art: discrete logic circuits with logic gate circuits used to realize logic functions for digital signals, application-specific integrated circuits with suitable combinational logic gate circuits, programmable gate arrays (PGAs), field programmable gate arrays (FPGAs), etc.

Those of ordinary skill in the art can understand that implementation of all or a part of the steps in the method of the foregoing embodiments can be completed by a program that instructs relevant hardware. The program may be stored in a computer-readable storage medium. The program can include one of or a combination of the steps of the method embodiment.

In addition, all functional units in all the embodiments of the present disclosure can be integrated into one processing module, or each unit can physically exist alone, or two or more units can be integrated in one module. The above integrated modules can be implemented in the form of hardware, or can be implemented in the form of software functional modules. The integrated module, if implemented in the form of a software functional unit and sold or used as a standalone product, may be stored in a computer readable storage medium.

The above-mentioned storage medium may be a read-only memory, a magnetic disk or an optical disk, and the like.

In the description of the present specification, descriptions of the reference terms such as “one embodiment”, “some embodiments”, “examples”, “specific examples,” or “some examples” mean that specific features, structures, materials or characteristics described in combination with the embodiments or examples are included in at least one embodiment or example of the present disclosure. In this specification, schematic representations of the above terms do not necessarily refer to the same embodiment or example. Furthermore, the particular features, structures, materials or characteristics described may be combined in any suitable manner in any one or more embodiments or examples.

Although the embodiments of the present disclosure have been shown and described above, it can be understood that the above embodiments are exemplary and should not be construed as limiting the present disclosure. Those of ordinary skill in the art can make changes, modifications, substitutions, and variations to the above-mentioned embodiments within the scope of the present disclosure.

What is claimed is:

1. A rapid genetic screening method, comprising:
receiving a sample to be tested, and lysing the sample to be tested at a high temperature to obtain cell lysate including a nucleic acid, wherein the sample to be tested comprises a whole blood or saliva or nasopharyngeal swab or wound swab sample of a patient;
performing isothermal amplification on the cell lysate by means of a loop-mediated isothermal amplification (LAMP) principle to obtain amplified nucleic acid liquid product;
fusing the nucleic acid liquid product with a preset biosensor, wherein the biosensor is pre-provided with a DNA probe which is only bounded to a specific nucleic acid segment, and after the DNA probe is bounded to the specific nucleic acid segment, the biosensor changes a self impedance value;
inputting an electrical signal to an input end of the biosensor, and collecting signal data of an output end of the biosensor; and
determining, on the basis of the signal data, whether the sample to be tested contains a nucleic acid segment matched with the DNA probe.

2. The rapid genetic screening method according to claim 1, wherein the determining, on the basis of the signal data, whether the sample to be tested contains a nucleic acid segment matched with the DNA probe comprises:
constructing a machine-learning algorithm model;
extracting a characteristic value of the signal data of the output end of the biosensor by means of a characteristic extraction algorithm combined with preset principal component analysis and a support vector machine; and
determining, by means of a preset machine-learning algorithm model and the characteristic value, whether the sample to be tested contains a nucleic acid segment matched with the DNA probe.

3. The rapid genetic screening method according to claim 1, further comprising:
judging, by means of a preset hyperplane dichotomy algorithm, a determination result of whether the sample to be tested contains a nucleic acid segment matched with the DNA probe.

4. The rapid genetic screening method according to claim 1, further comprising:
eliminating, on the basis of a preset algorithm model, signal noise generated by impurities in the nucleic acid liquid product.

5. A rapid genetic screening device, comprising: a micro-fluidic chip, a biosensor, and a portable testing main body, wherein the micro-fluidic chip is detachably inserted on the portable testing main body; the biosensor is arranged on the micro-fluidic chip;
the micro-fluidic chip comprises a first chamber, a second chamber, and a third chamber; the first chamber is used for accommodating a sample to be tested; the second chamber is used for performing loop-mediated isothermal amplification (LAMP) reaction; the third chamber is provided with the biosensor and is used for fusion of nucleic acid liquid product with the biosensor; a first micro valve is arranged between the first chamber and the second chamber; a second micro valve is arranged between the second chamber and the third chamber;
the portable testing main body comprises a main control module, and an interface module, a temperature control module, a power control module, a frequency generation module, a data storage module, and a signal processing/AI algorithm module which are all connected to the main control module;
the main control module is connected to the micro-fluidic chip through the interface module, and controls, by means of the temperature control module, a temperature of each chamber of the micro-fluidic chip; flowing of liquid inside the micro-fluidic chip and opening and closing of the micro valves are controlled by means of the power control module; an electrical signal is generated by the frequency generation module and is input into the biosensor; signal data of an output end of the biosensor is stored and recorded by means of the data storage module; and the signal data of the output end of the biosensor is analyzed by means of the signal processing/AI algorithm module.

6. The rapid genetic screening device according to claim 5, wherein the power control module comprises a motor driving sub-module, a micro pump sub-module, and a flow control sub-module;
the motor driving sub-module, the micro pump sub-module, and the flow control sub-module are all connected to the main control module.

7. The rapid genetic screening device according to claim 5, wherein the interface module comprises a plurality of USB interfaces;
the plurality of USB interfaces is used for being simultaneously connected to a plurality of micro-fluidic chips.

8. The rapid genetic screening device according to claim 7, further comprising a testing chip gating/control module, wherein
the main control chip selects and controls, through the testing chip gating/control module, the micro-fluidic chip connected to the portable testing main body.

9. The rapid genetic screening device according to claim 5, further comprising a communication module, wherein
the communication module is connected to the main control module and is used for transmitting data to external equipment.

10. The rapid genetic screening device according to claim 5, further comprising a human-computer interaction module.
